(19) United States
(12) Patent Application Publication
(10) Pub. No.: US 2024/0099203 A1
(43) Pub. Date: Mar. 28, 2024

(54) SENSOR ARRANGEMENT

(71) Applicant: LIGHT SCIENCE TECHNOLOGIES LIMITED, Derbyshire (GB)

(72) Inventors: John Matcham, Derbyshire (GB); Craig Price

(21) Appl. No.: 18/256,465

(22) PCT Filed: Dec. 8, 2021
(86) PCT No.: PCT/GB2021/000136

§ 371 Date: Jun. 8, 2023

(30) Foreign Application Priority Data
Dec. 8, 2020 (GB) 2019299.3

(51) Int. Cl. A01G 9/26; G01N 33/00

(52) U.S. Cl. CPC A01G 9/26; G01N 33/0098; G01N 2033/245

(57) ABSTRACT

The disclosure relates to a sensor arrangement for a controlled agriculture environment for growing plants. The sensor arrangement comprises a modular housing having first and second modules that are releasably coupled to one another. The first module is associated with a plant stem zone and has at least one sensor configured to sense a parameter associated with the plant stem zone conditions. The second is associated with a plant root zone and has at least one sensor configured to sense a parameter associated with the plant root zone conditions. The disclosure further relates to a controlled environment agriculture system comprising a plurality of such sensor arrangements that are connected to a management system.

### RELATED APPLICATIONS

The present application is a US National Phase entry of PCT/GB2021/000136 filed Dec. 8, 2021, which claims priority to Great Britain Application No. 2019299.3 filed Dec. 8, 2020, the entirety of which are incorporated herein by reference.

### FIELD OF THE DISCLOSURE

The present disclosure relates to controlled environment agriculture and more specifically to a sensor arrangement for use in a controlled environment agriculture system.

### BACKGROUND

Controlled environment agriculture (CEA) is a form of intensive farming where certain parameters affecting plant growth are closely monitored and or managed to optimise yield and/or revenue. Typical CEA environments are for example greenhouses and polytunnels. Although efforts have been made in the field of CEA, current systems still have many shortcomings holding back yield and/or revenue optimization. One of the challenges facing CEA is the fact there are a large number of variables interacting with one another leading to very few conditions being the same within comparable, or even the one, CEA installation(s). Data collection is an important part of CEA, but existing sensor arrangements can be complex, costly, not adaptable enough to serve different CEA installations, or unable to cope well with differing conditions within a single CEA installation.

It is clear that components and systems currently in use have multiple disadvantages associated with them and the current disclosure is aimed at overcoming at least some of these disadvantages.

### SUMMARY

According to a first aspect there is provided a sensor arrangement for a controlled agriculture environment for growing plants. The sensor arrangement comprises a modular housing having first and second modules. The first module is associated with a plant stem zone and has at least one sensor configured to sense a parameter associated with the plant stem zone conditions. The second module is associated with a plant root zone and has at least one sensor configured to sense a parameter associated with the plant root zone conditions.

In a second aspect there is provided a controlled environment agriculture (CEA) system comprising a gateway and a group of sensor arrangements. The sensor arrangements in the group are configured to communicate with one another and one of the sensor arrangements in the group is configured to communicate with the gateway on behalf of the group.

### BRIEF DESCRIPTION OF THE DRAWINGS

Embodiments will now be described by way of example only, with reference to the Figures, in which:

FIG. 1 is a diagrammatic representation of a sensor arrangement having first and second modules in accordance with the current disclosure;

FIG. 2 is a diagrammatic representation of the sensor arrangement of FIG. 1 with the first and second modules decoupled;

FIG. 3 is a bottom view of the first module of FIG. 1;

FIG. 4 is an isometric view of the first module of FIG. 1;

FIG. 5 is a top view of the first module of FIG. 1;

FIG. 6 is a bottom view of the second module of FIG. 1;

FIG. 7 is an isometric view of the second module of FIG. 1;

FIG. 8 is a top view of the second module of FIG. 1;

FIG. 9 is a diagrammatic functional representation of the first and second modules of FIG. 1.

FIGS. 10-14 are alternative exemplary embodiments of the sensor arrangement shown in FIG. 1;

FIG. 15 is a diagrammatic representation of a communication arrangement for a plurality of sensor arrangements in accordance with the current disclosure;

FIG. 16 is a diagrammatic representation of an alternative communication arrangement to the one shown in FIG. 15;

FIG. 17a-c are diagrammatic representations of exemplary communication arrangements between modules in a sensor arrangement;

FIGS. 18-20 are diagrammatic representations showing power and date flows between modules in a sensor arrangement;

FIG. 21 is a diagrammatic cut-away representation of an exemplary embodiment of a module associated with plant stem zone conditions;

FIG. 22 is a diagrammatic representation of an exemplary embodiment for seal arrangement between modules of the sensor arrangement;

FIGS. 23a-b are diagrammatic representations of an exemplary electric connection between modules in a sensor arrangement (in connected and disconnected states respectively);

FIG. 24 is a cut-away representation of an exemplary embodiment of a module associated with plant stem zone conditions;

FIG. 25 is a disc-shaped member for closing the module shown in FIG. 24;

FIG. 26 FIG. 24 is a cut-away representation of an exemplary embodiment of a module associated with plant root zone conditions;

FIG. 27 is an exemplary embodiment of a combined ground spike/sensor arrangement for the module of FIG. 26.

### DETAILED DESCRIPTION

Aspects and embodiments of the present disclosure will now be discussed with reference to the accompanying figures. Further aspects and embodiments will be apparent to those skilled in the art.

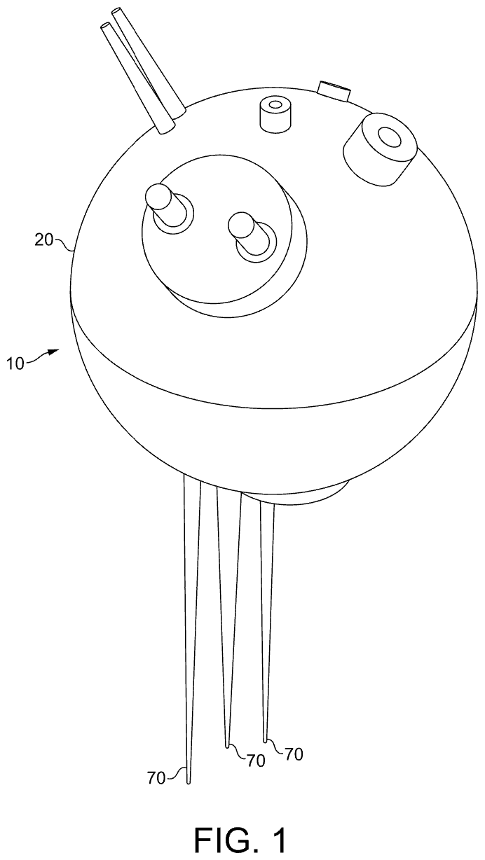

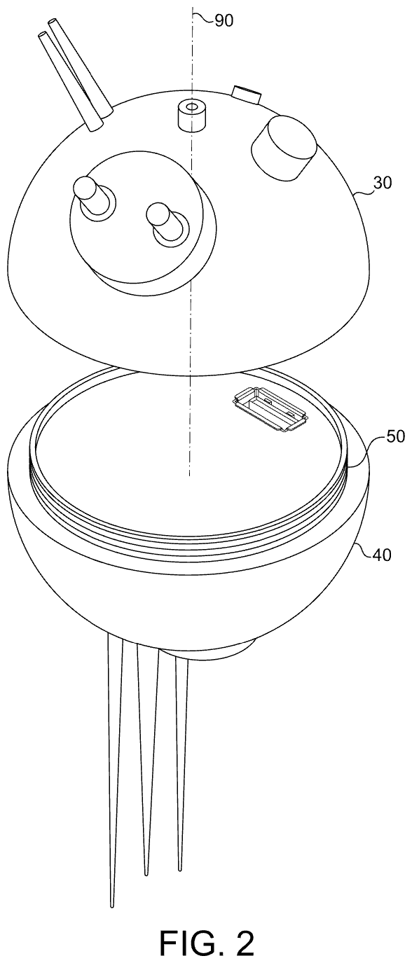

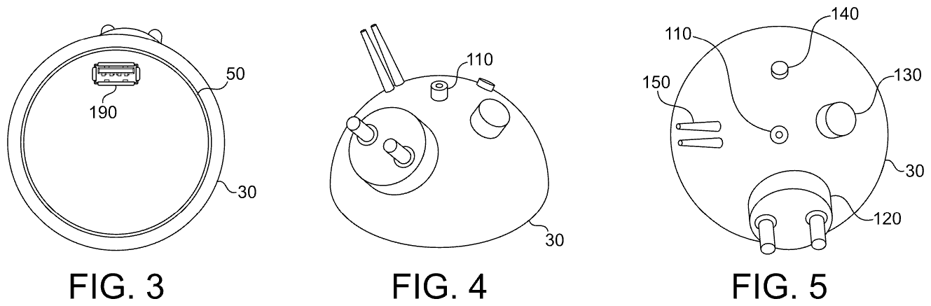

FIG. 1 shows an exemplary embodiment of a sensor arrangement 10 in accordance with the current disclosure. The sensor arrangement 10 is configurable to operate in a variety of situations and is particularly suited to operate in a controlled environment agriculture (CEA) setting to optimise plant growth. The sensor arrangement 10 may be part of a CEA management system 15 (not shown). The CEA management system 15 may include a plurality of sensor arrangements 10. CEA means any environment where at least some parameters affecting plant growth can to a certain extent be controlled. Often this entails growing plants in some form of permanent or temporary structure, using technologies such as hydroponics, aeroponics, aquaculture, and aquaponics. Examples of CEA environments are greenhouses, polytunnels, urban agriculture and vertical farms. The plants may grow roots in any suitable growing medium, such as gaseous, liquid, or solid media or substrates.

The sensor arrangement 10 includes a modular housing 20. The modular housing 20 may be of any suitable shape such as for example a generally spherical, cylindrical, dome, (frusto-) conical, or cuboid shape. In an embodiment the modular housing 20 has at least a first module 30 and a second module 40. In another embodiment the modular housing includes a third module 42. In another embodiment the modular housing 20 includes a fourth module 47. The modular housing 20 may include further modules if desirable. In some embodiments one or more of the modules 30, 40, 42, 47 may be omitted. A protective cap 49 may be provided if one of the modules is omitted.

At least some of the modules 30, 40, 42, 47 may be coupled to one another via a releasable coupling arrangement 50. The coupling arrangement 50 enables the modules 30, 40, 42, 47 to be separated from one another as can be seen for example in FIG. 2. The coupling arrangement 50 may include any suitable coupling type such as for example a threaded arrangement, a bajonet-style fitting, or an interference fit. The coupling 50 may be provided with a sealing arrangement 60, to, for example, prevent ingress of dust or humidity. The sealing arrangement 60 may include a resilient sealing member 62 and/or a skirt 52. In an embodiment the skirt 52 may be a substantially circumferential projection from one module that overlaps a portion of an adjacent module to protect the coupling 50 from ingress under gravity.

The sensor arrangement 10 is configured to operate in the vicinity of one or more plants with the first module 30 being predominantly associated with plant stem zone conditions and the second module 40 being predominantly associated with plant root zone conditions. The plant root zone may include the lower stem of the plant. In other words, the second module 40 tends to carry sensors (see below) associated with conditions in the growing medium and the first module 30 tends to carry sensors (see below) associated with conditions external to the growing medium. In an embodiment the third module 42 may be configured to sense parameters associated with air movement. In an embodiment the sensor arrangement 10 may include two modules 42, the first module 42 being adapted to sense air movement parameters near the stem zone and the second module 42 being adapted to sense air movement parameters near the root zone.

The sensor arrangement 10 may be positioned near, adjacent or amongst one or more plants. Depending on the growing medium and other factors it may be desirable to configure the housing 20 such that it can float on the growing medium or be fixed to the growing medium or any other suitable mounting point via a mechanical fixing such as for example one or more ground spikes 70 or a suspension or retaining chain 80 (not shown). It may be desirable to position at least a portion of the sensor arrangement 10 above or near the top of the vegetation to obtain more accurate sensor readings (e.g. light sensing) and/or the transmission of data.

The first module 30 may be provided with at least one sensor associated with plant stem zone conditions. FIGS. 4 and 5 show an exemplary external configuration of sensors on the first module 30, including a light sensor 110, a CO₂ sensor 120, a humidity sensor 130, a temperature sensor 140 and an airflow sensor 150. Some of these sensors 110-150 may be omitted. In an alternative embodiment, the airflow sensor 150 may be incorporated in the third module 42 instead of the first module 30 with an air passage 155 enabling air to flow to the airflow sensor 150. The light sensor 110 may be mounted to the first module 30 such that it can operate with rotational invariance. It may for example be located on an axis of rotational invariance 90 such as for example on the crown of the first module 30 as most clearly seen in FIGS. 4 and 5. In the exemplary embodiment as shown in FIGS. 4 and 5 the first module 30 is generally dome shaped with the top of the dome being the crown. In alternative embodiments the first module 30 may be of another suitable shape and the crown on those embodiments would be the (approximate) functional equivalent of the crown on the dome shape as shown in FIGS. 4 and 5. The other sensors 120-150 may be positioned on the first module 30 as preferred. In an embodiment at least a portion of at least one sensor may be positioned on an external surface of the first module, such as for example the light detecting portion of the light sensor 110.

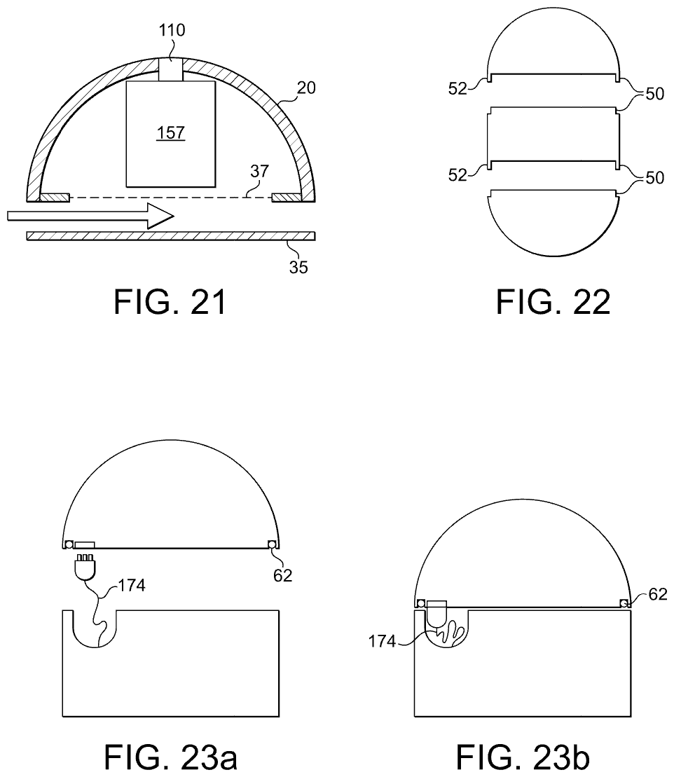

An alternative embodiment as shown for example in FIG. 21, comprises a predominantly internal configuration of sensors in the first module 30. A cluster 157 including at least some of the sensors 110-150 is located in the first module 30. Some of the sensors (e.g. the light sensor 110) my at least partially protrude through the wall of the first module 30. The light sensor 110 may be mounted to the first module 30 such that it can operate with rotational invariance. An air passage 35 enables air to reach the cluster 157. A permeable screen 37 may be provided to protect the internal environment of the first module 35 from environmental factors such as debris, plants and small animals (e.g. insects). The screen 37 may be incorporated into a generally disc-shaped member 38 that can be mounted to (and thereby form part of) the first module 30. A sealing arrangement 39 may be provided between the generally disc shaped member 38 and the first module 30.

The first module 30 may further include a power source 170. In an alternative embodiment, the power source 170 may be incorporated in the fourth module 47 instead of the first module 30. The first module 30 may include a communication arrangement 180. e first module 30 may further include a hardware port 190.

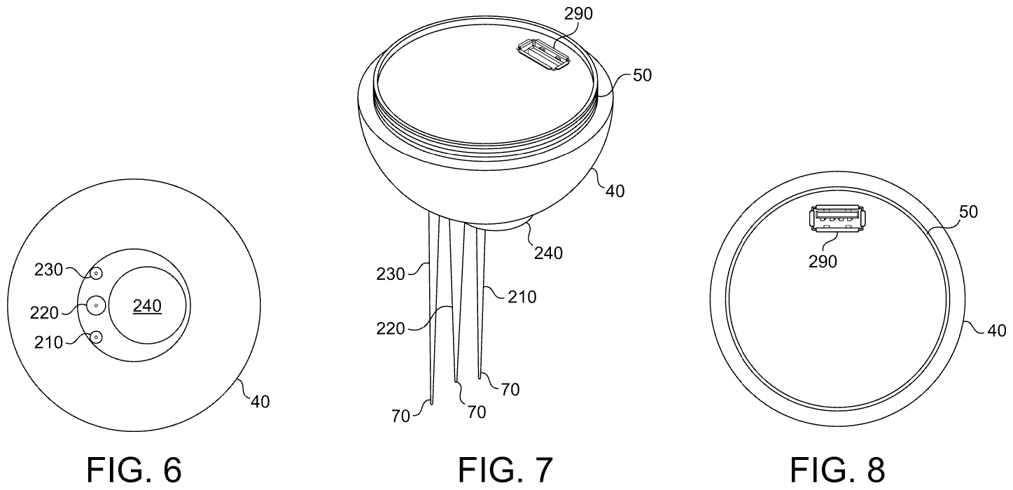

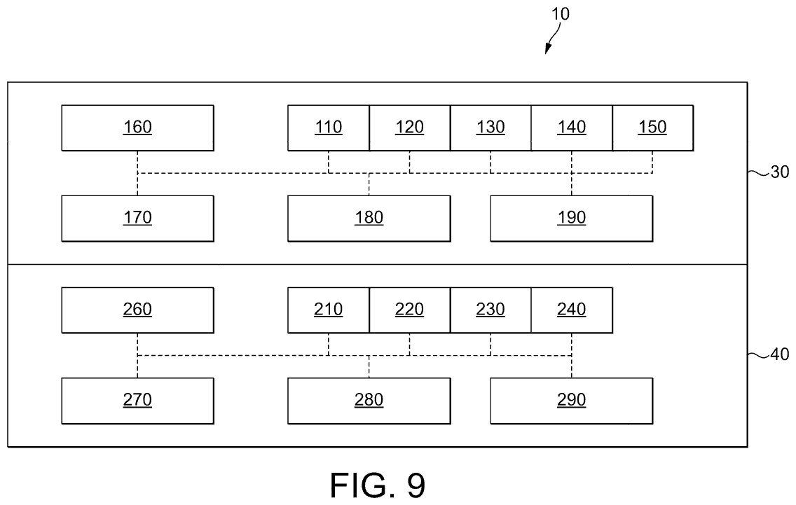

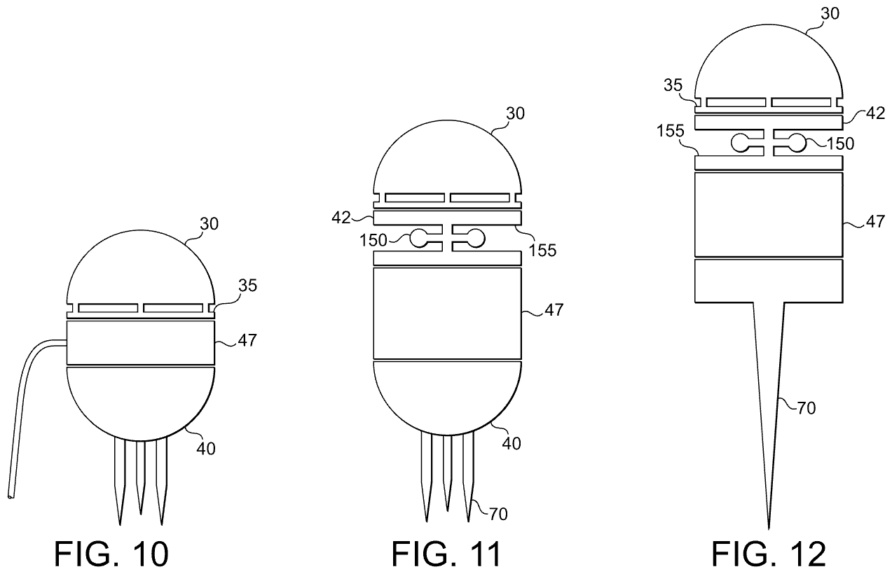

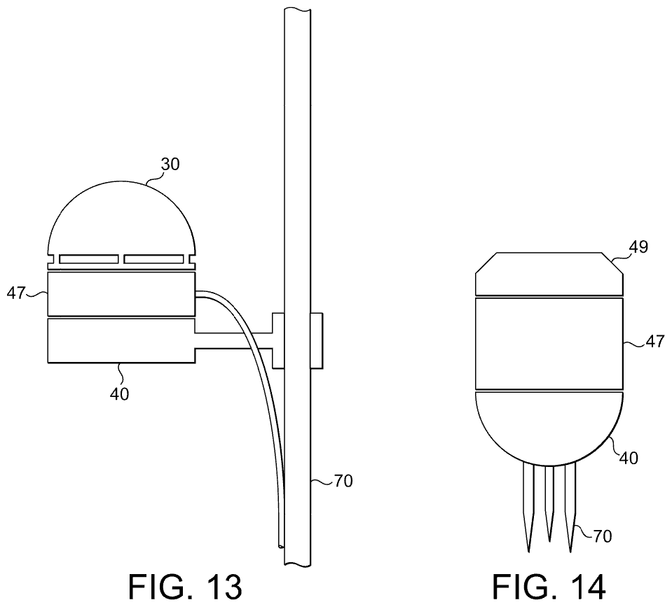

The second module 40 may be provided with at least one sensor associated with plant root zone conditions. FIGS. 6 and 7 show an exemplary configuration of sensors on the second module 40, including a temperature sensor 210, a nutrient sensor 220, a humidity sensor 230 and an oxygen sensor 240. Some of the sensors 210-240 may be omitted. One or more of the sensors 210-240 may double up as a ground spike 70. Alternatively, or additionally, a separate ground spike 70 without sensors may be provided.

The second module 40 may further include a power source 170. In an alternative embodiment, the power source 170 for the second module may be incorporated in the fourth module 47 instead of the second module 40. The second module 40 may include a communication arrangement 180. The second module 40 may include a hardware port 190.

A single, or multiple, power source(s) 170 may be used to power the first, second and third modules (30, 40, 42) where required. A secondary power source 170 may be provided to back up power supply in case of interrupted power supply from the primary power source 170.

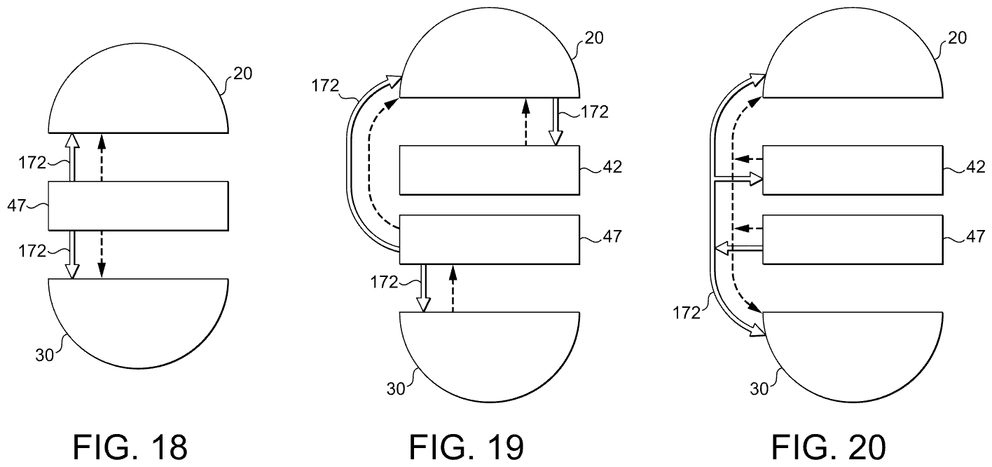

Power may flow between the various modules via a power bus 172. In an embodiment as shown in FIGS. 18 and 20, power may for example flow from the power source directly to a module. In another exemplary embodiment as shown in FIG. 19 power may flow from the power source indirectly to a module as it flows through another module first. The power bus 172 may be included in a joint connector 174 that may also include a portion of the communication arrangement 180. The power source 170 may be a rechargeable battery or an adapter receiving power from a mains or low power grid. The power source 170 may be rechargeable via wired or wireless technology and may be receiving a charge from a grid or from a renewable source such as solar or wind power.

The power source(s) may include a power source status system enabling a transmission of data indicating power source status between the various modules and to the gateway.

Exemplary data paths of power source status indication are shown as intermittent arrows in FIGS. 18-20.

A single, or multiple, control system(s) 160 may be used to control the first, second, third and fourth modules (20, 40, 42, 47). Where multiple control systems 160 are provided in one sensor arrangement 10, the multiple control systems 160 may communicate via the communication arrangement 180. The system 260 may include one or more processors and memory devices capable of storing and operating software to operate the sensor arrangement 10 and/or CAE control system 15.

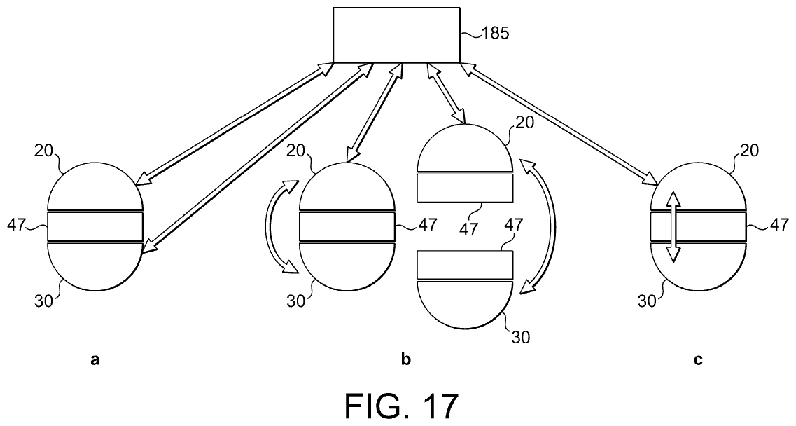

A single, or multiple, communication arrangement(s) 180 may be used to send and/or receive data as best seen in FIGS. 17a-c. A gateway 185 may be in communication with each single module 30, 40, 42, 47 or a selection thereof.

In an embodiment as shown in FIG. 17c, communication between multiple modules in one sensor arrangement 10 can take place via an internally wired arrangement and one of the modules can communicate with the gateway 185 on behalf of the various modules.

In an alternative embodiment as shown in FIG. 17b, communication between multiple modules in one sensor arrangement 10 can take place via a wireless arrangement (whether those modules are close coupled or spaced apart) and one of the modules can communicate with the gateway 185 on behalf of the various modules.

In an alternative embodiment as shown in FIG. 17a, each module communicates directly with the gateway 185.

A combination of any of the embodiments shown in FIGS. 17a-17c may be selected if desired.

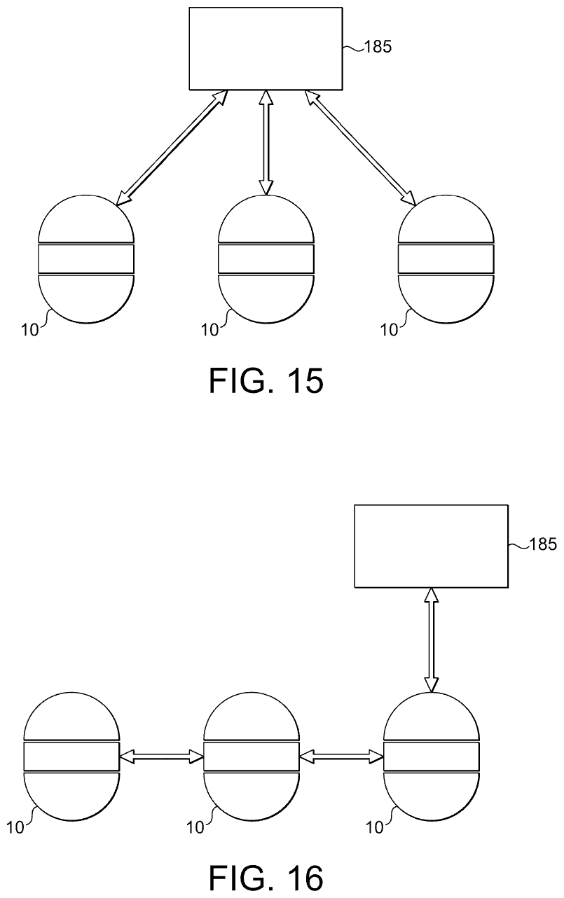

As shown in FIG. 15, a plurality of sensor arrangements 10 may each communicate directly with the gateway 185.

Alternatively, as shown in FIG. 16, a mesh arrangement may be used wherein a group of sensor arrangements 10 may communicate with each other with one sensor arrangement 10 communicating with the gateway 185 on behalf of the group of the sensor arrangements 10. Such mesh arrangement may enable a larger distance between the gateway 185 and the more remote sensor arrangements 10.

The communication arrangement 180 and any connection to the gateway 185 may include wired or wireless technologies such as WiFi, radio, or Bluetooth.

A single, or multiple, hardware ports 190 may be may be provided to any or all of the first, second, third and fourth modules (30, 40, 42, 47) where required. The hardware port 190 may be used for communication, data transfer, recharging the power source 170 and/or for software updates. The hardware port 190 may be a USB port.

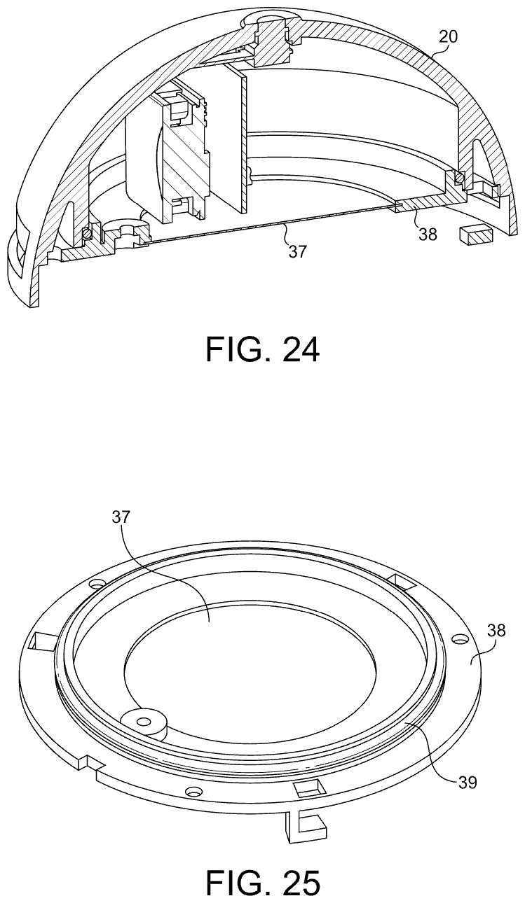

As shown in FIG. 24, at least one circuit board 300 carrying some components of at least one of the control and communication arrangements 160, 180 may be mounted in the first module 30. The circuit board 300 may be oriented generally vertically, or at least sufficiently angled, to prevent pooling of condensation on the circuit board.

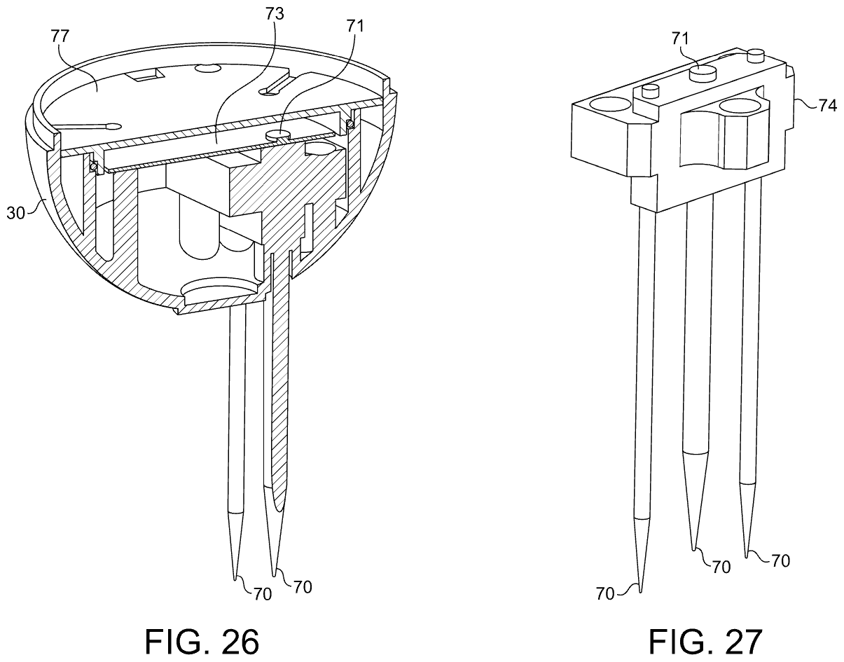

In the exemplary embodiment shown in FIG. 26, at least one of the ground spikes 70 functions as a sensor and the top 71 of the at least one ground spike 70 connects directly to a circuit board 73 in the second module 40. Other ground spikes 70 may also be attached directly to the circuit board 73.

The one or more ground spikes 70 may be set into a rigid holder 74 made of a suitable material such as plastic, to provide accurate spacing between the ground spikes 70 and/or to provide structural rigidity. The second module 40 may be closed off with a lid 77.

### INDUSTRIAL APPLICABILITY

In use, the sensor arrangement 10 may be placed near, adjacent or amongst plants. A plurality of sensor arrangements 10 may be used spread over a wider area. The sensor arrangement 10 may be configured for specific applications and may be provided with one, several, or all of the sensors 110-150, 210-240. In an exemplary embodiment the sensor arrangement may be provided with all the sensors 110-150 and 210-240. For example, the sensors 110-150 and 210-240 may sense the following:
light sensor 110 may sense a parameter indicative of light intensity and/or frequency.
CO₂ sensor 120 may sense a parameter indicative of CO₂ concentration
humidity sensor 130 may sense a parameter indicative of relative humidity
temperature sensor 140 may sense a parameter indicative of ambient air temperature
airflow sensor 150 may sense a parameter indicative of airflow (speed and/or direction)
temperature sensor 210 may sense a parameter indicative of root zone temperature
nutrient sensor 220 may sense one or more parameters indicative of nutrient levels in the root zone
humidity sensor 230 may sense one or more parameters indicative of water presence in the root zone
oxygen sensor 240 may sense one or more parameters indicative of root zone oxygen saturation.

The sensor arrangement 10 may float on the growing medium (e.g. water), be suspended (from the roof structure for example) or may be fixed onto the growing medium (e.g. soil) or on another element such as a plant tray or table. At least one of the sensors 210-240 may form, or be part of, the ground spike 70 such that the ground spike 70 can both support the sensor arrangement 10 on the growing medium and sense parameters in the growing medium.

During installation and operation it may be desirable to have the performance of the sensor arrangement 10 being rotationally invariant such that performance of the sensor arrangement 10 is not, or only mildly, affected by a certain positioning. This will facilitate a simpler and more cost effective installation and may contribute to system reliability. Some of the sensors 110-150, 210-240 may not need to be oriented or positioned in a specific manner, but some may have specific requirements. It may for example be important to have the same sensors on a plurality of sensor arrangements 10 all positioned the same way for a consistent approach to measurements. For example, it may be desirable to have all light sensors 110 face the artificial lighting installation often found in CEA environments and the light sensors may therefore for example all have to be facing upwards. Placing the light sensor 110 on the rotational axis of invariance 90 will enable an installer to quickly install the sensor arrangement 110 with fewer concerns about a specific rotational orientation of the sensor arrangement 110.

The sensor arrangement 10 may communicate data via gateway 185 to the CEA management system 15. The CEA management system 15 may use the data from one or more sensor arrangement(s) 10, to determine the conditions near a plant or group of plants. The CEA management system 15 may trigger an action to adjust conditions if the conditions are not in a preferred range. Actions may for example include adjusting one or more of temperature, airflow, humidity, water supply, nutrient dosing and light conditions. The CEA management system 15 may adjust one or more conditions throughout the whole of the CEA environment or at a local level only. The CEA management system 15 may also trigger an action in relation to a power source 170 if the power source status indication system indicates an adjustment, repair or replacement is desirable.

It will be understood that the invention is not limited to the embodiments above-described and various modifications and improvements can be made without departing from the concepts described herein. Except where mutually exclusive, any of the features may be employed separately or in combination with any other features and the disclosure extends to and includes all combinations and sub-combinations of one or more features described herein.

1. A sensor arrangement for a controlled agriculture environment for growing plants, the sensor arrangement comprising:
a modular housing comprising first and second modules,
the first module being associated with a plant stem zone and having at least one sensor configured to sense a parameter associated with the plant stem zone conditions,
the second module being associated with a plant root zone and having at least one sensor configured to sense a parameter associated with the plant root zone conditions.

2. A sensor arrangement according to claim 1, further comprising a third module configured to sense a parameter indicative of airflow.

3. A sensor arrangement according to claim 2, further comprising a fourth module configured as a power source for at least one of the first, second and third modules.

4. A sensor arrangement according to claim 3 wherein at least two of the first, second, third and fourth modules are releasably coupled to one another.

5. (canceled)

6. (canceled)

7. A sensor arrangement according to claim 1 wherein at least a portion of at least one sensor is positioned on an external surface of the first module.

8. A sensor arrangement according to claim 1 wherein at least one sensor is positioned on the inside of the first module and the first module is provided with a permeable screen to enable air to pass to that sensor.

9. A sensor arrangement according to claim 1 wherein the at least one sensor of the first module senses a parameter indicative of one or more of the following plant stem zone conditions:
a. CO2
b. Light
c. Humidity
d. Temperature
e. Airflow

10. A sensor arrangement according to claim 1 wherein the at least one sensor is a light sensor mounted to the first module such that it can operate with rotational invariance.

11. (canceled)

12. (canceled)

13. A sensor arrangement according to claim 1 wherein the least one sensor of the second module senses a parameter indicative of one or more of the following plant root zone conditions:
a. Root zone temperature
b. Nutrients
c. H₂O
d. Root zone oxygen

14. A sensor arrangement according to claim 1 wherein the second module is provided with at least one ground spike.

15. A sensor arrangement according to claim 14 wherein the at least one ground spike is a sensor.

16. A sensor arrangement according to claim 15 wherein the second module houses a circuit board and the at least one ground spike extends into the second module and connects directly to the circuit board.

17. A sensor arrangement according to claim 1 wherein the modular housing is configured to float.

18. A sensor arrangement according to claim 1 wherein the first module houses a circuit board orientated substantially vertically to prevent pooling of condensation.

19. A sensor arrangement according to claim 1 wherein at least one of the modules comprises a hardware port configured for at least one of:
a. recharging the power source
b. communication
c. data transmission
d. software updates

20. A sensor arrangement according to claim 4,
wherein at least two modules receive power directly from the fourth module.

21. A sensor arrangement according to claim 4,
wherein a module receives power directly from the fourth module and at least a portion of that power flows on to another module in the sensor arrangement.

22. (canceled)

23. A sensor arrangement according to claim 3 wherein the sensor arrangement further comprises a secondary power source to back up power supply in case of interrupted power supply from the fourth module.

24. A sensor arrangement according to claim 3 wherein at least two of the modules in a sensor arrangement are configured to communicate with one another and one of the modules is configured to communicate with another sensor arrangement or a gateway on behalf of the at least two modules.

25. A controlled environment agriculture (CEA) system comprising a gateway and a group of sensor arrangements according to claim 1, wherein the sensor arrangements in the group are configured to communicate with one another and one of the sensor arrangements in the group is configured to communicate with the gateway on behalf of the group.
